(19) United States
(12) Patent Application Publication
(10) Pub. No.: US 2019/0076108 A1
(43) Pub. Date: Mar. 14, 2019

(54) BREAST IMAGING APPARATUS, DOSE CALCULATING APPARATUS, CONTROL METHOD FOR BREAST IMAGING APPARATUS, DOSE CALCULATING METHOD, AND NON-TRANSITORY COMPUTER-READABLE MEDIUM

(71) Applicant: CANON KABUSHIKI KAISHA, Tokyo (JP)

(72) Inventor: Yoshihito Machida, Sagamihara-shi (JP)

(21) Appl. No.: 16/122,167

(22) Filed: Sep. 5, 2018

(30) Foreign Application Priority Data
Sep. 11, 2017 (JP) 2017-174246

(51) Int. Cl. A61B 6/00; A61B 6/02; G06T 17/00; G06T 11/00

(52) U.S. Cl. CPC A61B 6/542; A61B 6/02; A61B 6/502; A61B 6/466; G06T 2210/41; A61B 6/5235; G06T 17/00; G06T 11/008; A61B 6/5217

(57) ABSTRACT

A breast imaging apparatus obtains tomographic images of the breast of an object. The apparatus includes an obtaining unit configured to obtain imaging conditions when the tomographic images are obtained, a generating unit configured to generate a breast model including a specific tissue with respect to the breast of the object, and a calculating unit configured to calculate the dose of radiation absorbed by the specific tissue by a radiation imaging simulation using the imaging conditions and the breast model.

### BACKGROUND OF THE INVENTION

### Field of the Invention

The present invention relates to a breast imaging apparatus, a dose calculating apparatus, a control method for the breast imaging apparatus, a dose calculating method, and a non-transitory computer-readable medium.

### Description of the Related Art

A breast imaging apparatus has a function of outputting the actual performance records concerning average mammary gland doses after breast imaging to manage radiation exposure doses. Japanese Patent Laid-Open No. 2009-100926 discloses a method of obtaining an average mammary gland dose based on a correction coefficient for each pixel with respect to a difference from 50% of the mammary gland content ratio obtained for each pixel and correction coefficients with respect to materials for an anode and a radiation quality filter. Japanese Patent Laid-Open No. 2017-047103 discloses, as an imaging condition setting unit for a breast tomography apparatus, a method of obtaining imaging conditions before an imaging operation from the three-dimensional shape of the breast and mammary gland content ratios.

Conventional techniques, however, have given no consideration to the three-dimensional distribution of the mammary gland in the breast when outputting the actual performance records concerning doses at the time of breast tomography, and hence have had difficulty in calculating accurate mammary gland doses.

### SUMMARY OF THE INVENTION

According to embodiments of the present invention, a breast imaging apparatus which can calculate accurate mammary gland doses is provided.

According to one aspect of the present invention, there is provided a breast imaging apparatus that obtains a tomographic image of a breast of an object, the apparatus comprising: an obtaining unit configured to obtain an imaging condition when the tomographic image is obtained; a generating unit configured to generate a breast model including a specific tissue with respect to the breast of the object; and a calculating unit configured to calculate a dose of radiation absorbed by the specific tissue by a radiation imaging simulation using the imaging condition and the breast model.

According to another aspect of the present invention, there is provided a dose calculating apparatus comprising: an obtaining unit configured to obtain a tomographic image of an object and an imaging condition when the tomographic image is obtained; a generating unit configured to generate a three-dimensional model with a three-dimensional area of a specific tissue of the object and a three-dimensional area other than the specific tissue being discriminated; and a calculating unit configured to calculate a dose of radiation absorbed by the specific tissue based on the imaging condition and the three-dimensional model.

According to another aspect of the present invention, there is provided a control method for a breast imaging apparatus that obtains a tomographic image of a breast of an object, the method comprising: obtaining an imaging condition when the tomographic image is obtained; generating a breast model including a three-dimensional model of a specific tissue with respect to the breast of the object; and calculating a dose of radiation absorbed by the specific tissue by a radiation imaging simulation using the imaging condition and the breast model.

According to another aspect of the present invention, there is provided a dose calculating method comprising: obtaining a tomographic image of an object and an imaging condition when the tomographic image is obtained; generating a three-dimensional model with a three-dimensional area of a specific tissue of the object and a three-dimensional area other than the specific tissue being discriminated; and calculating a dose of radiation absorbed by the specific tissue based on the imaging condition and the three-dimensional model.

According to another aspect of the present invention, there is provided a non-transitory computer-readable medium storing a program for causing a computer to execute a control method for a breast imaging apparatus configured to obtain a tomographic image of a breast of an object, the control method comprising: obtaining an imaging condition when the tomographic image is obtained; generating a breast model including a three-dimensional model of a specific tissue with respect to the breast of the object; and calculating a dose of radiation absorbed by the specific tissue by a radiation imaging simulation using the imaging condition and the breast model.

According to another aspect of the present invention, there is provided a non-transitory computer-readable medium storing a program for causing a computer to execute a dose calculating method, the dose calculating method comprising: obtaining a tomographic image of an object and an imaging condition when the tomographic image is obtained; generating a three-dimensional model with a three-dimensional area of a specific tissue of the object and a three-dimensional area other than the specific tissue being discriminated; and calculating a dose of radiation absorbed by the specific tissue based on the imaging condition and the three-dimensional model.

Further features of the present invention will become apparent from the following description of exemplary embodiments (with reference to the attached drawings).

### BRIEF DESCRIPTION OF THE DRAWINGS

FIG. 1A is a block diagram showing an example of the functional arrangement of a breast tomography apparatus according to the first embodiment;

FIG. 1B is a flowchart showing the operation of the breast tomography apparatus according to the first embodiment;

FIG. 2A is a block diagram showing an example of the functional arrangement of a breast model generation unit according to the first embodiment;

FIG. 2B is a flowchart showing a processing procedure in the breast model generation unit;

FIGS. 3A to 3C are views each showing an example of a tomographic image according to the first embodiment;

FIG. 4 is a graph showing an example of mammary gland and adipose linear attenuation coefficients according to the first embodiment;

FIG. 5A is a block diagram showing an example of the functional arrangement of a simulation condition generation unit according to the first embodiment;

FIG. 5B is a flowchart showing a processing procedure in the simulation condition generation unit according to the first embodiment;

FIGS. 6A and 6B are views showing an example of setting a geometric arrangement by the simulation condition generation unit;

FIG. 7 is a graph showing an example of a spectrum according to the first embodiment;

FIG. 8A is a block diagram showing an example of the functional arrangement of a mammary gland dose calculating unit according to the first embodiment;

FIG. 8B is a flowchart showing a processing procedure performed by the mammary gland dose calculating unit;

FIG. 9 is a flowchart showing a processing procedure in a mammary gland absorbed energy calculating unit according to the first embodiment;

FIG. 10A is a block diagram showing an example of the functional arrangement of a mammary gland dose calculating unit according to modification 1-2;

FIG. 10B is a flowchart showing a processing procedure in the mammary gland dose calculating unit;

FIG. 11A is a view showing an example of overlaying a mammary gland dose distribution on a tomographic image;

FIG. 11B is a view showing an example of designating an exclusion area by an exclusion area designation unit;

FIG. 12A is a block diagram showing an example of the functional arrangement of a breast model generation unit according to the second embodiment;

FIG. 12B is a flowchart showing a processing procedure in the breast model generation unit according to the second embodiment;

FIGS. 13A and 13B are views showing how a tomographic image according to the second embodiment is reduced;

FIG. 14 is a view showing count examples of absorbed photons according to the second embodiment;

FIG. 15 is a view showing count examples of absorbed photons according to modification 2-1;

FIG. 16A is a view showing an example of dividing a breast model into seven areas;

FIG. 16B is a view showing an example of dividing a breast model into four areas; and

FIG. 17 is a block diagram showing an example of the arrangement of a breast tomography apparatus according to an embodiment.

### DESCRIPTION OF THE EMBODIMENTS

The embodiments of the present invention will be described below by referring to the accompanying drawings as needed. Although each embodiment will exemplify a case in which the tomographic images obtained by a breast CT apparatus are used, the present invention can also be applied to a breast tomosynthesis apparatus and other types of tomography apparatuses.

### First Embodiment

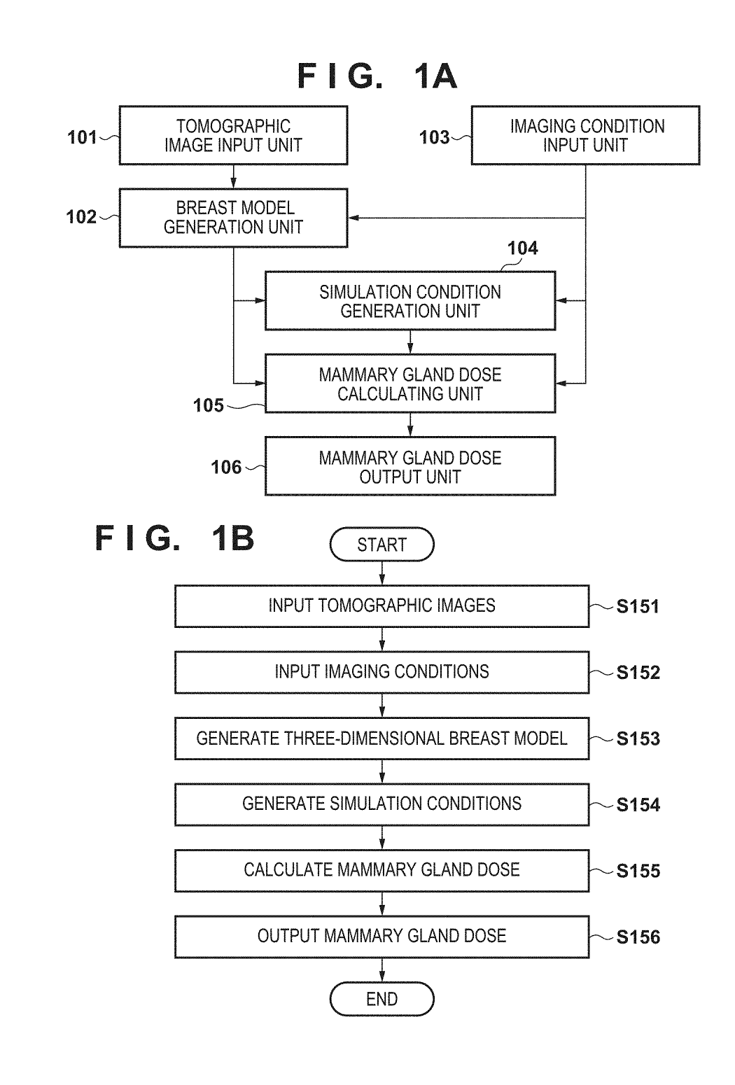

The first embodiment will exemplify a breast imaging apparatus (to be referred to as a breast tomography apparatus 100) that obtains a tomographic image of the breast of an object by using FIGS. 1A to 9 and 17.

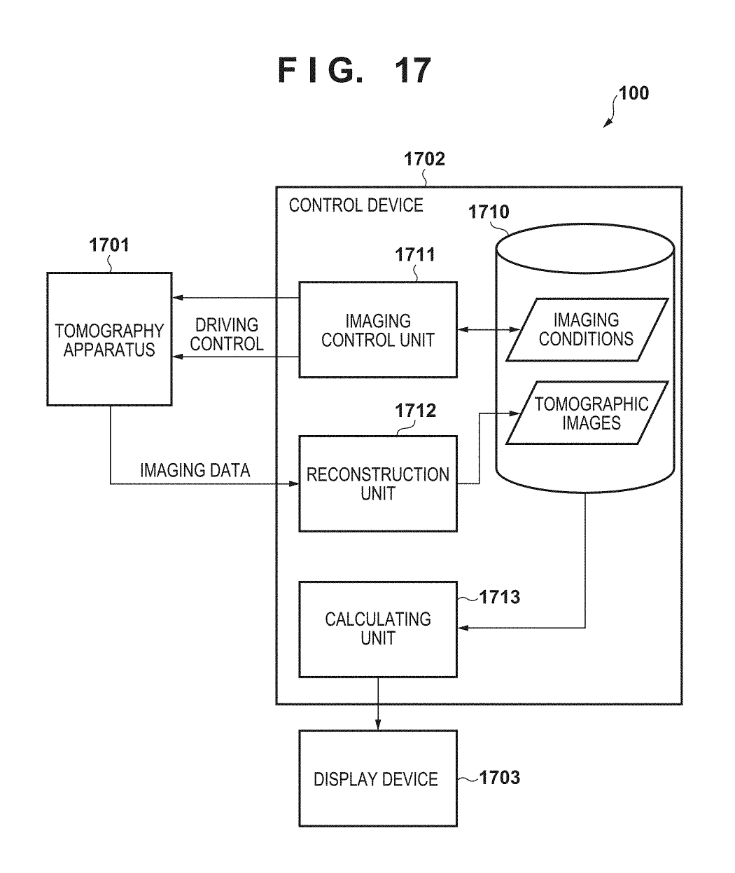

The arrangement of the breast tomography apparatus 100 will be described first with reference to FIG. 17. The tomography apparatus is a breast CT apparatus, and performs radiation imaging of the breast by driving control by an imaging control unit 1711. A control device 1702 reconstructs a tomographic image by controlling a tomography apparatus 1701. The imaging control unit 1711 performs driving control on the tomography apparatus based on imaging conditions stored in a storage unit 1710. A reconstruction unit 1712 reconstructs a tomographic image from image data obtained by the tomography apparatus 1701, and stores the tomographic image in the storage unit 1710. A calculating unit 1713 calculates mammary gland doses by simulation using imaging conditions and tomographic images and displays the calculated mammary gland doses on a display device 1703. The details of the arrangement of the calculating unit 1713 and a mammary gland dose calculating method will be described below. Note that the imaging control unit 1711, the reconstruction unit 1712, and the calculating unit 1713 may be implemented by making a CPU execute predetermined programs or may be partly or totally implemented by dedicated hardware.

FIG. 1A is a block diagram showing an example of the functional arrangement of the calculating unit 1713. As shown in FIG. 1A, the calculating unit 1713 includes a tomographic image input unit 101, a breast model generation unit 102, an imaging condition input unit 103, a simulation condition generation unit 104, a mammary gland dose calculating unit 105, and a mammary gland dose output unit 106. The operation of each unit of the calculating unit 1713 will be described below with reference to FIG. 1B.

In step S151, the tomographic image input unit 101 inputs a three-dimensional tomographic image of an object breast. For example, the tomographic image input unit 101 obtains a three-dimensional tomographic image by obtaining a plurality of tomographic images arrayed in a direction perpendicular to the slice direction from the storage unit 1710. In step S152, the imaging condition input unit 103 obtains imaging conditions when obtaining a tomographic image of the object breast. The imaging conditions are stored in the storage unit 1710. Note that the imaging conditions include, for example, a tube voltage, a radiation target material, the position/shape/material of an added filter, a cone angle, a fan angle, the position/shape/material of a collimator, a tube current, an irradiation time, a rotational speed, a projection count, the distance from a radiation focus to a rotation center, the distance from a radiation focus to the radiation detector, and an air kerma measured at the rotation center.

In step S153, the breast model generation unit 102 generates a breast model including a specific tissue with respect to the breast of an object. In this embodiment, the specific tissue is a mammary gland. More specifically, the breast model generation unit 102 generates a three-dimensional breast model including the breast interior by using the tomographic images of the object breast input in step S151 and the imaging conditions input in step S152. A three-dimensional breast model indicates a three-dimensional model area of at least the mammary gland and adipose. Note that a three-dimensional breast model generation method performed by the breast model generation unit 102 will be described in detail later. A three-dimensional breast model will be simply referred to as a breast model hereinafter.

In step S154, the simulation condition generation unit 104 generates simulation conditions based on the breast model generated in step S153 and the imaging conditions input in step S152. Note that a simulation condition generation method will be described in detail later.

In step S155, the mammary gland dose calculating unit 105 calculates the dose of radiation absorbed by a specific tissue (mammary gland) by a radiation imaging simulation using the imaging conditions and the breast model. More specifically, the mammary gland dose calculating unit 105 performs a simulation based on the simulation conditions generated in step S154 to calculate mammary gland doses that are the doses of radiation absorbed by the mammary gland. A mammary gland dose calculating method will be described in detail later. In step S156, the mammary gland dose output unit 106 outputs the mammary gland doses. In outputting the mammary gland doses, for example, this data may be stored in the storage unit 1710 as data to be displayed on the display device 1703 or stored in the storage unit 1710 together with image data.

The processing from steps S151 to S156 is performed in the above manner to generate a three-dimensional model of the breast from tomographic images of the object and calculate and output the doses of radiation absorbed by the mammary gland using a simulation, thereby accurately managing the exposure doses on the breast.

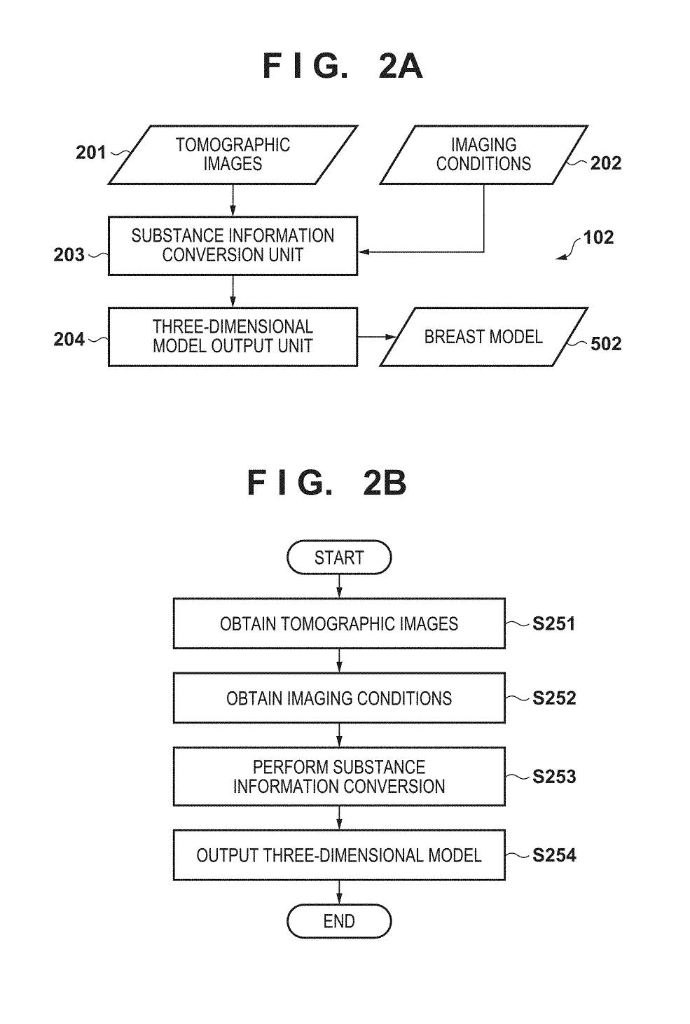

An object breast model generation method in the breast model generation unit 102 will be described next with reference to FIGS. 2A to 4. The breast model generation unit 102 generates a three-dimensional model of a specific tissue (mammary gland) from voxels, of the voxels constituting the three-dimensional tomographic image based on the tomographic images, which are classified as mammary gland voxels based on voxel values, and generates a breast model including the three-dimensional model. FIG. 2A is a block diagram showing the arrangement of the breast model generation unit 102. FIG. 2B is a flowchart showing the operation of the breast model generation unit 102. As shown in FIG. 2A, the breast model generation unit 102 includes a substance information conversion unit 203 and a three-dimensional model output unit 204, and generates a breast model 502 based on tomographic images 201 and imaging conditions 202 (radiation energy).

A processing procedure executed by the breast model generation unit 102 will be described in detail with reference to FIG. 2B. In step S251, the substance information conversion unit 203 obtains the tomographic images 201 constituting a three-dimensional tomographic image of the breast input from the tomographic image input unit 101. Note that the value of each pixel of the tomographic images 201 is based on a linear attenuation coefficient of the object. In general, a tomography apparatus linearly converts values based on linear attenuation coefficients into CT values, with −1000 representing air and 0 representing water. In this embodiment, for the sake of simplicity, the distribution of linear attenuation coefficients itself is regarded as a three-dimensional tomographic image.

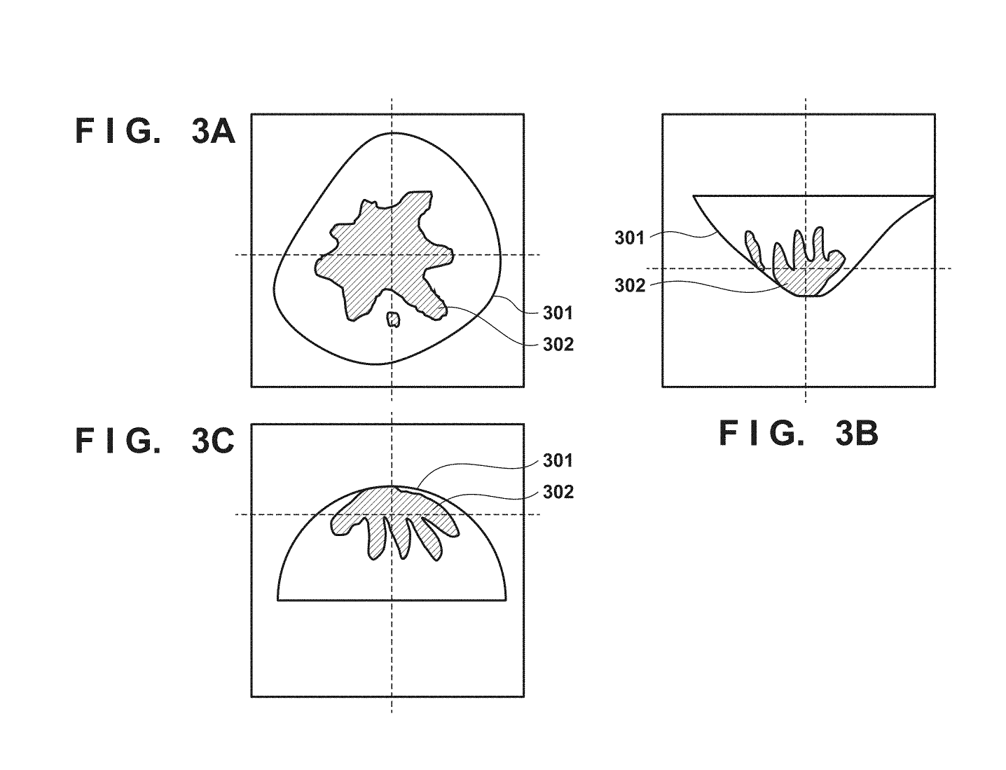

FIGS. 3A to 3C are views each showing an example of a tomographic image. FIG. 3A shows a coronal plane. FIG. 3B shows a transverse plane. FIG. 3C shows a sagittal plane. A line 301 indicates the skin surface of the breast, and the inside under the skin surface is filled with the mammary gland and the adipose. Assume that a hatched area 302 indicates values close to the linear attenuation coefficients of the mammary gland, the remaining portion indicates values close to the linear attenuation coefficient of the adipose. In an actual tomographic image, pixel values vary in some range depending on the influences of quantum noise and the like.

In step S252, the substance information conversion unit 203 obtains the imaging conditions 202 input by the imaging condition input unit 103. In this case, the substance information conversion unit 203 obtains the energy information of radiation associated with linear attenuation coefficients. In step S253, the substance information conversion unit 203 converts the pixels (to be referred to as voxels hereinafter) of each tomographic image into target substances based on the pixel values of the tomographic image 201 and the energy information of radiation of the imaging conditions 202. The following will exemplify a case in which the breast is separated into the mammary gland and the adipose.

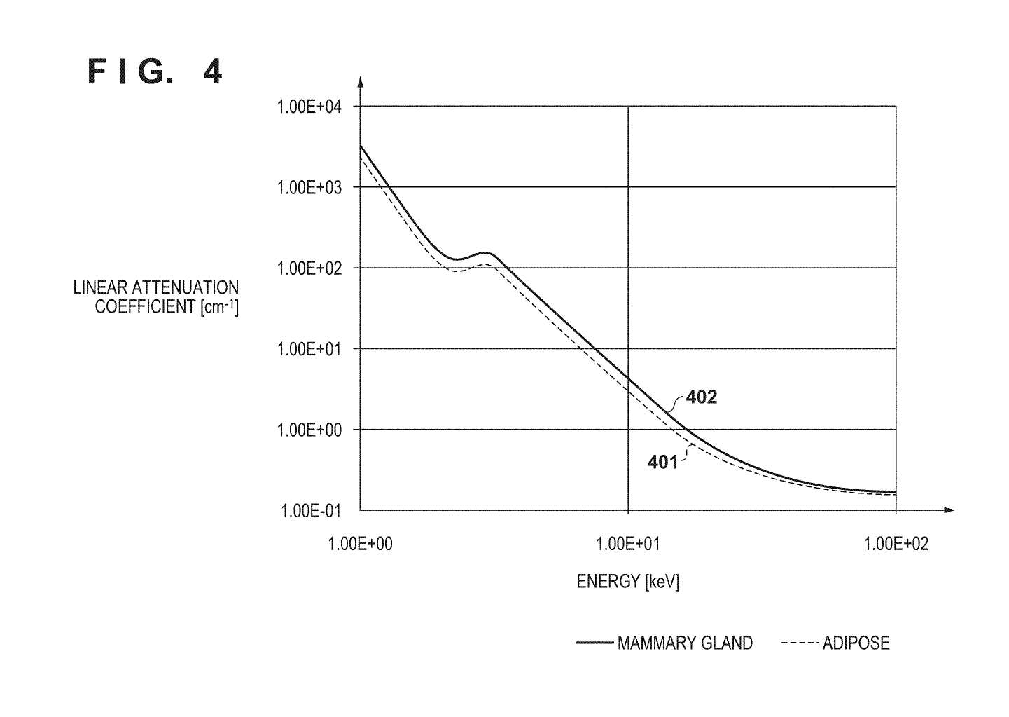

FIG. 4 is a graph showing the linear attenuation coefficients of the mammary gland and the adipose. In the graph, the abscissa represents the energies of radiation, and the ordinate represents the linear attenuation coefficients. A curve 401 represents the linear attenuation coefficients of the adipose. A curve 402 represents the linear attenuation coefficients of the mammary gland. The substance information conversion unit 203 obtains the linear attenuation coefficients of the mammary gland and the adipose based on the energies of radiation at the time of imaging, and decides each voxel in the tomographic image as a mammary gland or adipose voxel depending on to which substance the linear attenuation coefficient of the voxel is close. Note that the outside of the breast in a tomographic image is air. In this embodiment, for the sake of simplicity, a description of the air portion will be omitted. Assume, however, that the outside of the breast is filled with air. Note that the energy of radiation to be actually applied is not generally monoenergy. In this case, a substance whose linear attenuation coefficient is known, such as water, may be imaged in advance for each combination of a tube voltage, a target, and an added filter to obtain the energy of radiation corresponding to imaging conditions from the linear attenuation coefficient.

As described above, a three-dimensional model (called a breast model) of the breast is generated by classifying the respective voxels in the breast in a three-dimensional tomographic image into mammary gland voxels and adipose voxels. In step S254, the three-dimensional model output unit 204 outputs the generated three-dimensional model (breast model 502). Performing the processing from step S251 to step S254 described above can generate the breast model 502 as a three-dimensional model of the breast from tomographic images of the breast of the object.

Note that although the above embodiment has exemplified the case in which the breast is separated into the mammary gland and the adipose, the breast may be separated into the mammary gland, the adipose, the skin, an implant, a calcified part, and the like. Increasing the number of separated parts in this manner can generate a more accurate three-dimensional breast model and cope with various types of objects. In addition, introducing the skin into a model allows dose measurement on the skin.

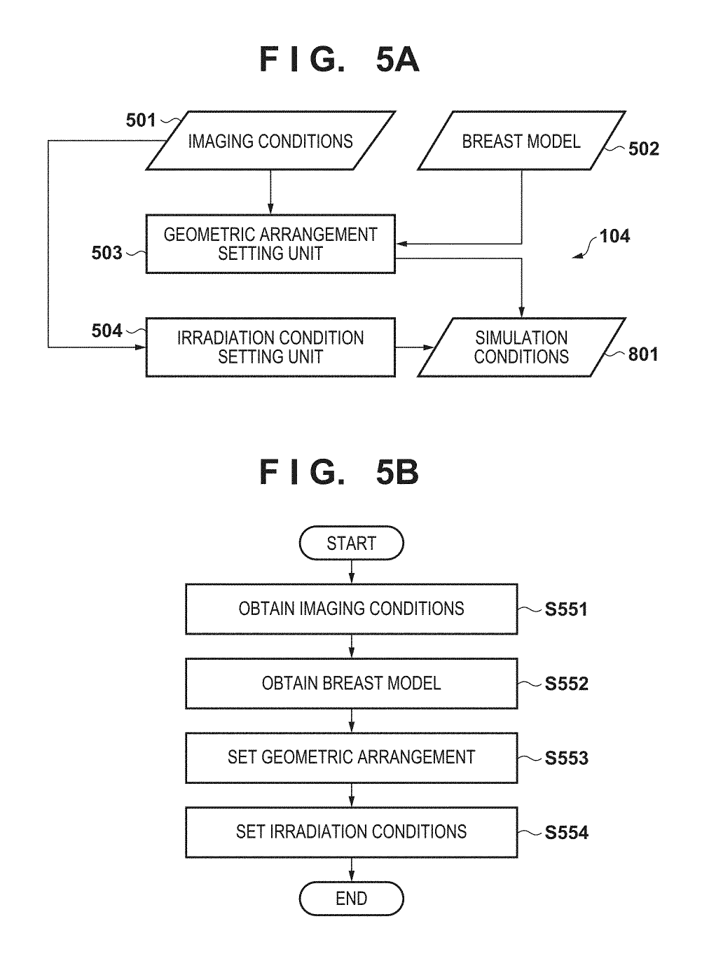

A simulation condition generation method in the simulation condition generation unit 104 will be described next with reference to FIGS. 5A to 7. FIG. 5A is a block diagram showing an example of the functional arrangement of the simulation condition generation unit 104. As shown in FIG. 5A, the simulation condition generation unit 104 includes a geometric arrangement setting unit 503 and an irradiation condition setting unit 504, and sets simulation conditions 801 by using imaging conditions 501 and the breast model 502.

A processing procedure executed by the simulation condition generation unit 104 will be described in detail next with reference to the flowchart of FIG. 5B. In step S551, the geometric arrangement setting unit 503 obtains the imaging conditions 501 input by the imaging condition input unit 103 in step S152 in FIG. 1B and stored in the storage unit 1710. The imaging conditions 501 are the same as those described above.

In step S552, the geometric arrangement setting unit 503 obtains the breast model 502 generated by the breast model generation unit 102. As described above, in the breast model 502, each voxel is discriminated as a breast voxel or an adipose voxel. Note that the central coordinates of this breast model coincide with the rotation center.

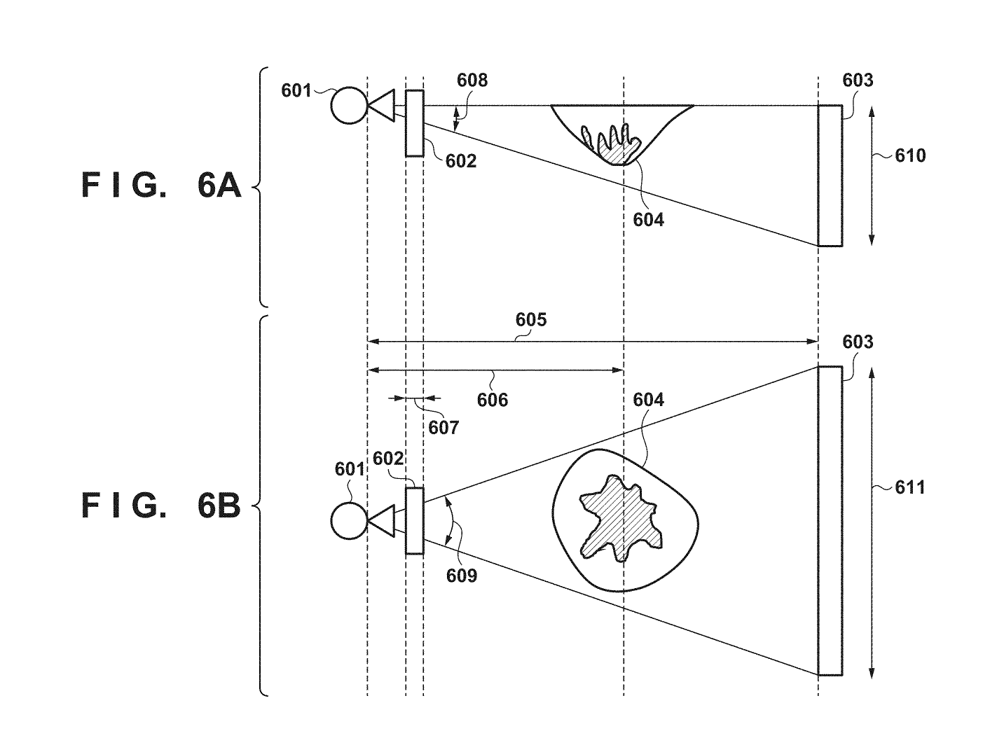

In step S553, the geometric arrangement setting unit 503 sets an initial geometric arrangement based on the imaging conditions and the breast model. FIGS. 6A and 6B show the initial geometric arrangement set by the geometric arrangement setting unit 503. FIG. 6A shows the geometric arrangement viewed from the horizontal direction of the object. FIG. 6B shows the geometric arrangement viewed from the chest-wall nipple direction of the object.

Referring to FIGS. 6A and 6B, reference numeral 601 denotes a reflectance irradiation unit; 602, an added filter; 603, a radiation detector; 604, the arrangement of the breast model 502; 605, the distance from the focus of the reflectance irradiation unit 601 to the detection surface of the radiation detector 603; 606, the distance from the focus of the reflectance irradiation unit 601 to the rotation center; 607, the thickness of the added filter 602; 608, the cone angle of radiation; 609, the fan angle of radiation; 610, the height of the radiation detector 603; and 611, the width of the radiation detector 603. As described above, the geometric arrangement setting unit 503 sets a geometric arrangement as the simulation conditions 801 by using the imaging conditions 501 and the breast model 502.

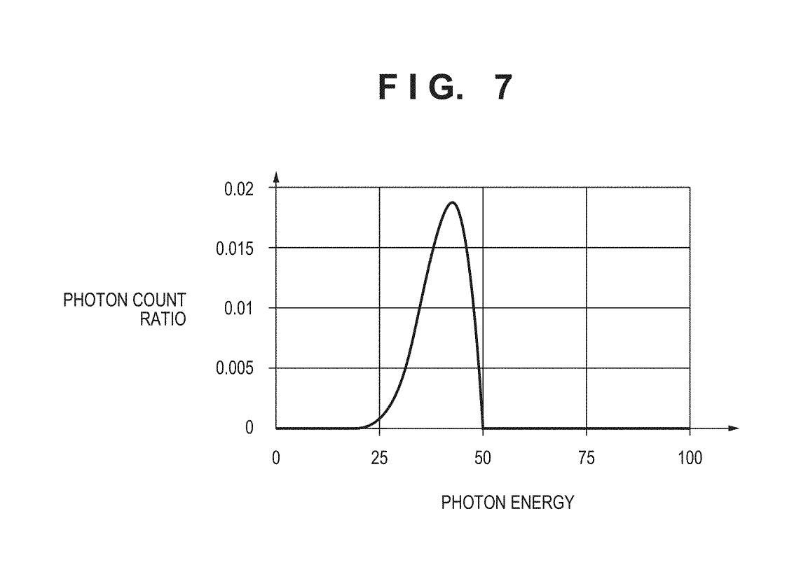

In step S554, the irradiation condition setting unit 504 sets irradiation conditions as the simulation conditions 801 based on the imaging conditions 501. The irradiation conditions set in this case include the spectrum of radiation to be applied, a photon count, and a projection count. FIG. 7 is a graph showing an example of the spectrum of radiation. In this graph, the abscissa represents the energies of radiation photons, and the ordinate represents the photon count ratios. Note that it is possible to use, as this spectrum, a spectrum actually measured in advance by a measurement device such as a spectrometer in accordance with a tube voltage, a tube current, and an irradiation time. In addition, when obtaining conditions such as a tube voltage and a target, the spectrum of radiation may be obtained by using Kramers' formula, Birch-Marshall formula, or the like.

A photon count may be set in accordance with a required accuracy and a required calculation time. The larger the photon count, the higher the accuracy of calculation results and the longer the calculation time. Note that such photon counts are distributed for the respective energies in accordance with the above radiation spectrum. In the first embodiment, a photon count setting method is set in accordance with a required accuracy and a required calculation time. However, when photon counts are known in advance, the same photon counts as those set at the time of actual imaging may be set.

As many a projection count as that set at the time of obtaining tomographic images may be set. Although in the first embodiment, as many a projection count as that set at the time of obtaining tomographic images is set, a simulation may be performed with a projection count smaller than that set at the time of obtaining tomographic images of the breast. That is, the projection count may be reduced in accordance with a required accuracy, a required calculation time, and the like. Assume that the projection count is reduced to ½. In this case, when the projection count at the time of imaging is 360 and the angular intervals between the projections are 1°, the set projection count is set 180, and the angular intervals between the projections are set to 2°. Setting a projection count smaller than the projection count at the time of imaging can shorten the required time for dose calculation (to be described later).

As described above, the simulation condition generation unit 104 sets the simulation conditions 801 by performing the processing from step S551 to step S554.

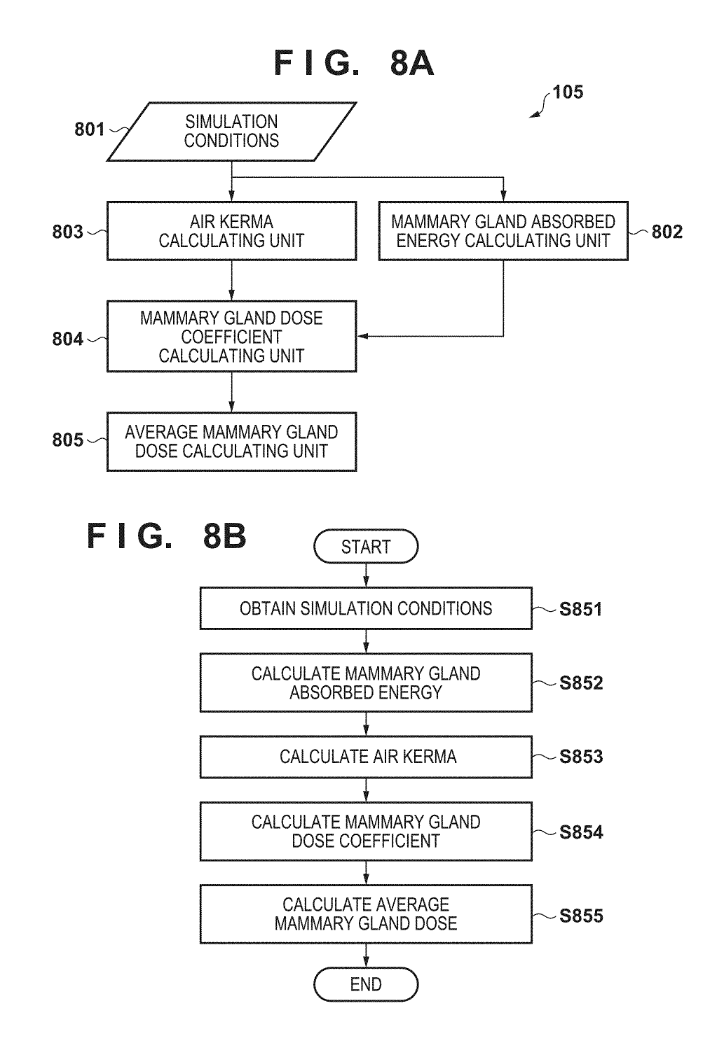

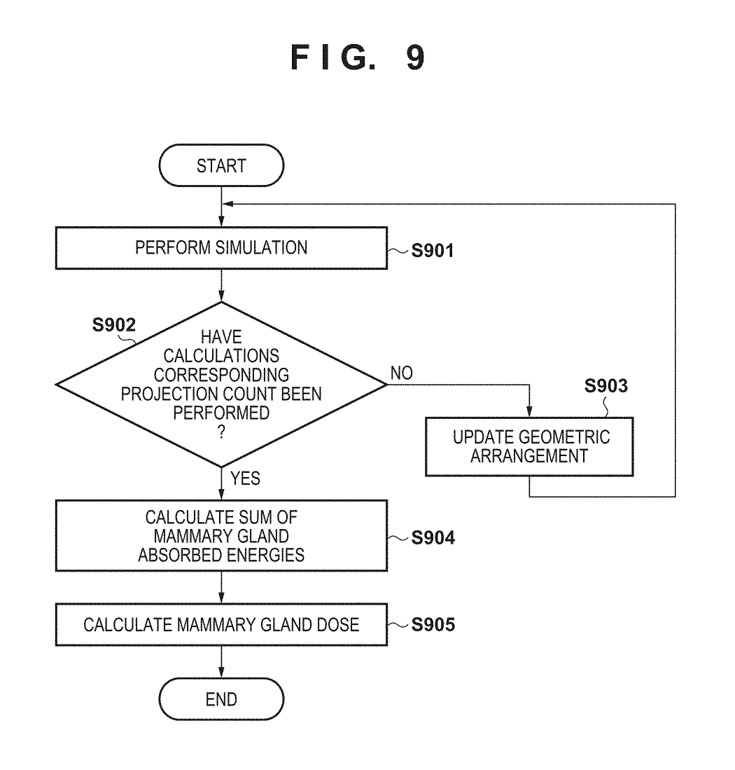

A mammary gland dose (average mammary gland dose) calculating method by using the mammary gland dose calculating unit 105 will be described next with reference to FIGS. 8A, 8B, and 9. FIG. 8A is a block diagram showing an example of the arrangement of the mammary gland dose calculating unit 105. As shown in FIG. 8A, the mammary gland dose calculating unit 105 includes a mammary gland absorbed energy calculating unit 802, an air kerma calculating unit 803, a mammary gland dose coefficient calculating unit 804, and an average mammary gland dose calculating unit 805, and calculates a mammary gland dose by using the simulation conditions 801.

A processing procedure executed by the mammary gland dose calculating unit 105 will be described in detail next with reference to FIG. 8B.

In step S851, the mammary gland absorbed energy calculating unit 802 and the air kerma calculating unit 803 obtain the simulation conditions 801 set by the simulation condition generation unit 104 described above. In step S852, the mammary gland absorbed energy calculating unit 802 obtains a mammary gland dose by a simulation by calculating the energy absorbed in the mammary gland. The unit of energy absorbed in the mammary gland is [mGy]. Note that a calculating method performed by the mammary gland absorbed energy calculating unit 802 will be described later.

In step S853, the air kerma calculating unit 803 calculates an air kerma at the time of simulation at the rotation center position of radiation applied under the simulation conditions 801. Note that the air kerma calculated in this case may differ from that of radiation applied at the time of obtaining a tomographic image or from that of radiation at the time of the above mammary gland dose calculation. For the sake of simplicity, assume that in this embodiment, an air kerma is calculated with the same photon count as that used by the mammary gland absorbed energy calculating unit 802. At this time, the unit of air kerma is [mGy].

In step S854, the mammary gland dose coefficient calculating unit 804 calculates a mammary gland dose coefficient for converting an air kerma into an average mammary gland dose. A mammary gland dose coefficient can be calculated by using equation (1):

$${D}_{g} {N}_{CT} = \frac{{D}_{gsim}}{{D}_{airsim}}$$

where D_{g}N_{CT} is a mammary gland dose coefficient, D_{gsim} is the mammary gland dose obtained by the above simulation, and Dₐᵢᵣₛᵢₘ is an air kerma at the time of the above simulation.

In step S855, the average mammary gland dose calculating unit 805 calculates an average mammary gland dose by using mammary gland dose coefficients and imaging conditions. An average mammary gland dose is calculated by using equation (2):

D_{g}=D_{g}N_{CT}×Dₐᵢᵣ

where D_{g} is a mammary gland dose, D_{g}N_{CT} is a mammary gland dose coefficient, and Dₐᵢᵣ is an air kerma. The air kerma is the one measured at the rotation center under the same conditions as those set at the time of obtaining a tomographic image. An air kerma is preferably obtained in advance.

Note that an air kerma is obtained as a measurement result at the rotation center. However, this is not exhaustive. For example, it is possible to use a measurement result from an area dosimeter arranged near a radiation irradiation unit. That is, the average mammary gland dose calculating unit 805 may calculate an average mammary gland dose from mammary gland dose coefficients and dose values obtained by the area dosimeter. In this case, the air kerma calculated in step S853 is arranged in the same manner. In addition, using the doses obtained by the area dosimeter can incorporate the variable ratios of doses in a calculation at the time of simulation in step S852.

As described above, performing the calculation from step S851 to step S855 can calculate mammary gland doses from a three-dimensional model of the breast. Using the simulation using a three-dimensional arrangement of the mammary gland in this manner can more accurately calculate the actual performance records concerning the absorbed doses of the mammary gland.

FIG. 9 is a flowchart showing a mammary gland absorbed energy calculating method in the mammary gland absorbed energy calculating unit 802 (step S852). First of all, in step S901, the mammary gland absorbed energy calculating unit 802 performs a radiation imaging simulation with an initial geometric arrangement under the simulation conditions 801. This calculation may be performed by using a technique using a Monte Carlo simulation like that disclosed in non-patent literature (Medical Physics 31(2), February 2004 P226-235). The mammary gland absorbed energy calculating unit 802 counts the number of photons of the photons applied at this time for each energy absorbed by the mammary gland in the breast model.

In step S902, the mammary gland absorbed energy calculating unit 802 checks whether simulations corresponding to the projection count have been performed. If simulations corresponding to the projection count have been performed, the process advances to step S904. If simulations corresponding to the projection count have not been performed, the process advances to step S903. In step S903, the mammary gland absorbed energy calculating unit 802 updates the geometric arrangement under simulation conditions. In this case, the mammary gland absorbed energy calculating unit 802 updates the geometric arrangement by changing the arrangement of the radiation irradiation unit, the added filter, and the radiation detection unit centered on the rotation center in accordance with the angular intervals between projections. After step S903, the process returns to step S901, in which the mammary gland absorbed energy calculating unit 802 performs a radiation imaging simulation with the changed geometric arrangement.

In step S904, the mammary gland absorbed energy calculating unit 802 calculates the sum F_{gsim} of energies absorbed by the mammary gland by using the counted photon count and the corresponding energies according to equation (3):

F_{gsim}=∫E×N(E)dE

where E is the energy of a photon, and N(E) is the number of photons absorbed by the mammary gland for each energy.

In step S905, the mammary gland absorbed energy calculating unit 802 calculates a mammary gland dose by a simulation based on equation (4) from the sum of energies of radiation absorbed by the mammary gland, and provides the mammary gland dose to the mammary gland dose coefficient calculating unit 804.

$${D}_{gsim} = α \times \frac{{F}_{gsim}}{{M}_{gsim}}$$

where F_{gsim} is the sum of energies of radiation absorbed by the mammary gland and M_{gsim} is the mass of the mammary gland. The mass of the mammary gland may be obtained by multiplying the sum of volumes of voxels classified as mammary gland voxels by the density [g/cm³] of the mammary gland. Note that as the density [g/cm³] of the mammary gland, for example, the value disclosed in NIST (https://www.nist.gov/) or the like may be used. In addition, α is a coefficient for the conversion of a unit into [mGy]. For example, when the unit of F_{gsim} is [keV] and the unit of M_{gsim} is [kg], α is 1.60218×10⁻¹³.

As described above, performing the processing from step S901 to step S904 can obtain a mammary gland dose from a three-dimensional model of the mammary gland. According to this embodiment, because a mammary gland dose is calculated in this manner based on the three-dimensional model of the mammary gland, that is, the three-dimensional distribution of the mammary gland, a more accurate average mammary gland dose can be calculated.

### Modification 1-1

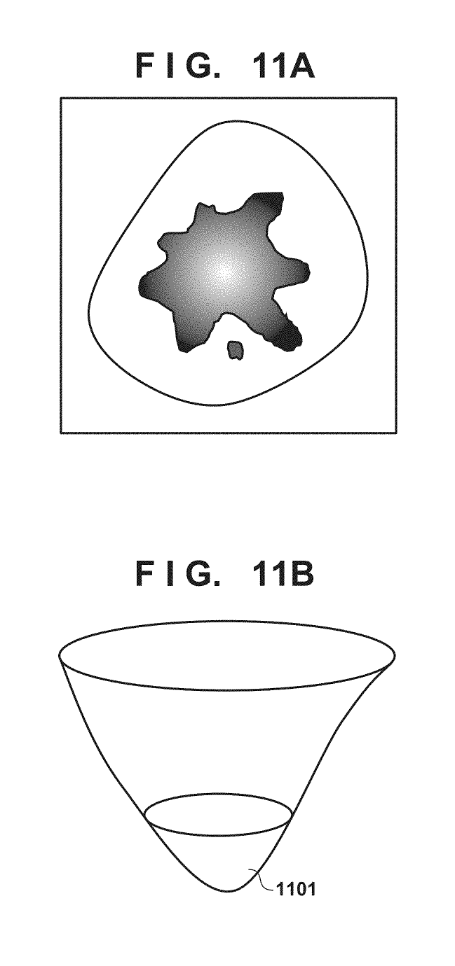

In the first embodiment, in step S156 (FIG. 1B), the mammary gland dose output unit 106 displays a mammary gland dose on the display, stores the mammary gland dose as data, and stores it together with image data. However, this is not exhaustive. For example, the distribution of mammary gland doses (energies absorbed by the mammary gland) calculated by the mammary gland dose calculating unit 105 may be displayed so as to be overlaid on a tomographic image. FIG. 11A shows an example in which modification 1-1 is applied to a coronal plane. Referring to FIG. 11A, in a mammary gland area, darker portions indicate larger amounts of energies absorbed by the mammary gland. Displaying data in this manner makes it possible to check the magnitude of a mammary gland dose at each portion in the mammary gland area. Note that in order to perform such display, it is necessary to calculate an absorbed dose for each of voxels classified as mammary gland voxels or each of partial areas obtained by dividing the mammary gland area.

### Modification 1-2

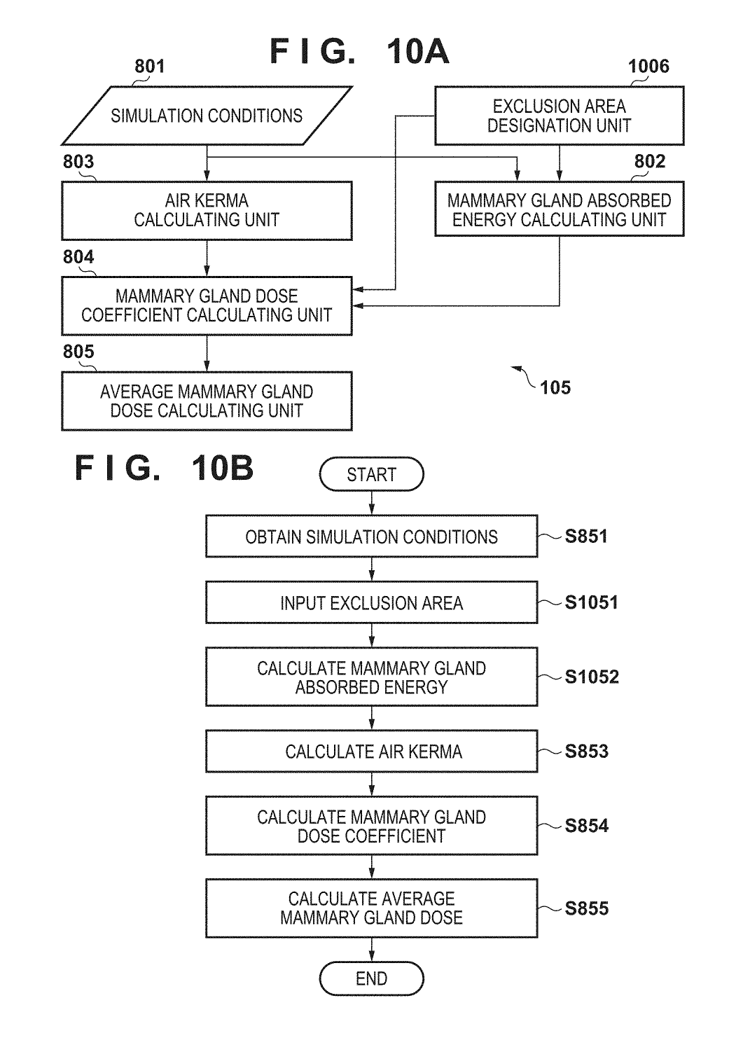

In the first embodiment, a mammary gland dose is calculated from the overall breast area. However, this is not exhaustive. A mammary gland dose may be calculated from a partial area of the object breast. FIG. 10A shows the arrangement of the mammary gland dose calculating unit 105 to which modification 1-2 is applied. As compared with the above embodiment (FIG. 8A), this unit additionally includes an exclusion area designation unit 1006. The mammary gland dose calculating unit 105 calculates a mammary gland dose from a breast model except for a portion designated by the exclusion area designation unit 1006.

FIG. 10B shows a procedure in a mammary gland dose calculating method in modification 1-2. In step S851, the air kerma calculating unit 803 and the mammary gland absorbed energy calculating unit 802 obtain the simulation conditions 801. In step S1051, the exclusion area designation unit 1006 designates an exclusion area. FIG. 11B shows an example of designating an exclusion area. FIG. 11B shows a three-dimensional image generated from tomographic image data, with an exclusion area 1101 being designated. In this manner, the exclusion area designation unit 1006 can designate an exclusion area from a three-dimensional image. In step S1052, the mammary gland absorbed energy calculating unit 802 calculates energy absorbed in a portion in the mammary gland excluding the exclusion area.

Subsequent steps S853, S854, and S855 are the same as those described in the first embodiment (FIG. 8B). Performing the above processing in FIG. 10B makes it possible to calculate a mammary gland dose in an area except for an exclusion area. Designating an exclusion area in this manner can manage a mammary gland dose except for a portion excised by a surgical operation or the like.

### Second Embodiment

The arrangement and basic operation of a breast tomography apparatus according to the second embodiment are the same as those of the first embodiment. A breast model generation method according to the second embodiment and a mammary gland dose calculating method using the breast model generation method will be described, mainly focusing on differences from the first embodiment. In the second embodiment, a breast model generation unit 102 generates a breast model with a small number of voxels by reducing tomographic image images. In addition, the breast model generation unit 102 generates a three-dimensional model of the mammary gland by using voxels classified as mixture voxels of mammary gland and adipose voxels based on voxel values. For example, in the second embodiment, the breast model generation unit 102 handles the overall breast as a mixture of the mammary gland and the adipose, calculates the mammary gland density for each voxel (from, for example, a ratio between the mammary gland and the adipose), and generates a breast model as the mixture of the mammary gland and the adipose. A mammary gland dose calculating unit 105 performs a simulation similar to that in the first embodiment based on the generated breast model, and performs calculation based on the mammary gland densities, thereby calculating a mammary gland dose. That is, the mammary gland dose calculating unit 105 calculates the absorbed dose of the mammary gland for each voxel based on the radiation absorbed dose of each voxel constituting the breast model and the mammary gland density as the ratio of the mammary gland and the adipose which is decided based on voxel values. In the second embodiment, it is possible to shorten the calculation time taken for a simulation by reducing a breast model.

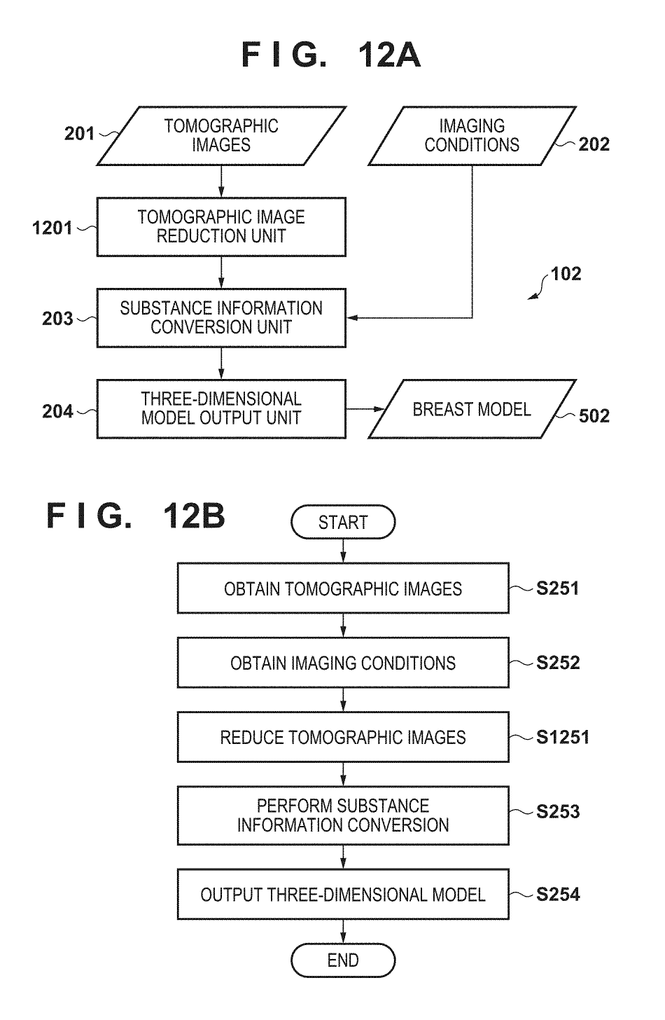

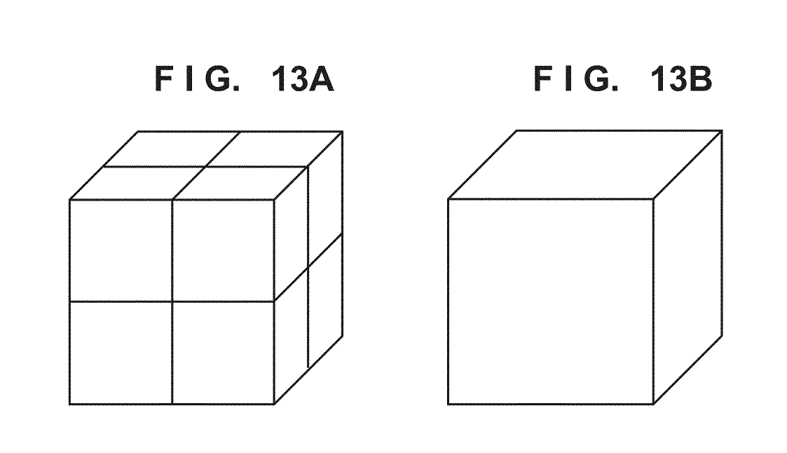

The arrangement of the breast model generation unit 102 and the breast model generation method according to the second embodiment will be described with reference to FIGS. 12A, 12B, 13A, and 13B. FIG. 12A is a block diagram showing the arrangement of the breast model generation unit 102. As shown in FIG. 12A, the breast model generation unit 102 includes a tomographic image reduction unit 1201, a substance information conversion unit 203, and a three-dimensional model output unit 204, and generates a breast model 502 based on tomographic images 201 and imaging conditions 202.

Processing executed by the breast model generation unit 102 will be described next with reference to the flowchart of FIG. 12B. In step S251, the tomographic image reduction unit 1201 obtains the tomographic images 201 input by the tomographic image input unit 101. In step S252, the substance information conversion unit 203 obtains the imaging conditions 202 input by an imaging condition input unit 103.

In step S1251, the tomographic image reduction unit 1201 reduces the voxel count of each tomographic image 201. The following is a case in which the voxel count is reduced to ⅛. FIGS. 13A and 13B are views for explaining a case in which eight voxels are reduced to one voxel. FIG. 13A shows original voxels. FIG. 13B shows a voxel after reduction. In order to reduce eight voxels to one voxel, the value of the voxel after reduction is equal to the sum (average value) of the values obtained by reducing the values of the original eight voxels to ⅛. Note that in the second embodiment, the value of a voxel after reduction is the average value of original voxels. However, this is not exhaustive. For example, the intermediate value or weighted average value of the values of voxels may be used. A breast model including the mixture of the mammary gland and the adipose is generated based on the values of voxels obtained by reducing tomographic images.

In step S253, the substance information conversion unit 203 converts the voxels of each tomographic image into target substances based on the voxel values of each reduced tomographic image and the imaging conditions 202 (radiation energy information). In this case, unlike in the first embodiment, target substances corresponding to linear attenuation coefficients are the mammary gland, the adipose, and their mixture. For example, when a linear attenuation coefficient is an intermediate value between the mammary gland and the adipose, the corresponding voxel is converted into a mixture voxel having a mammary gland density of 50% (0.5). In step S254, the three-dimensional model output unit 204 outputs the three-dimensional model calculated in the above manner as the breast model 502.

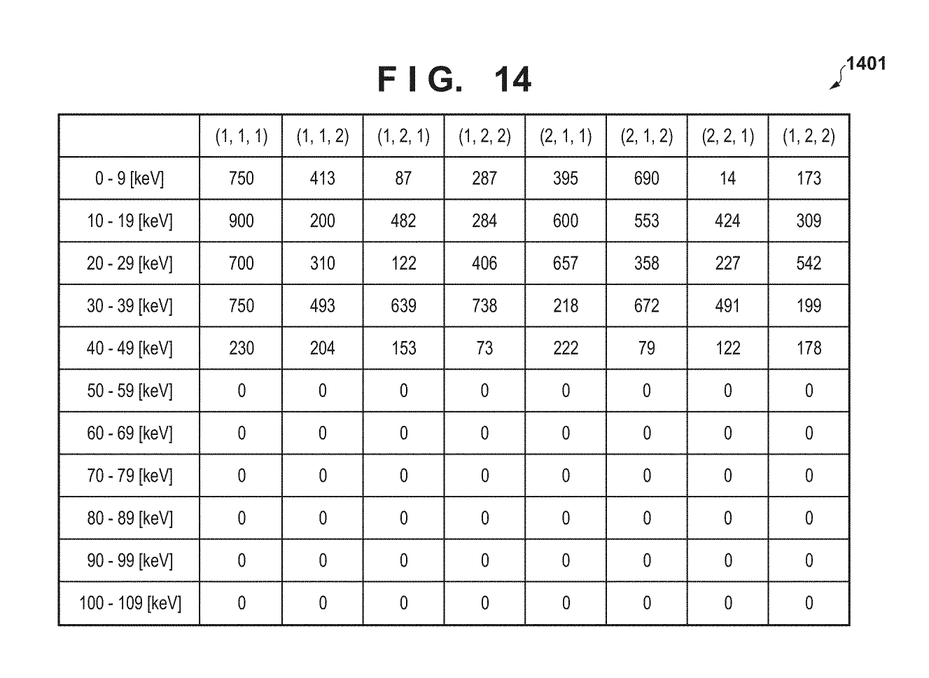

A mammary gland dose calculating method in the mammary gland dose calculating unit 105 will be described next with reference to the flowchart of FIG. 9 and FIG. 14. Note that the arrangement of the mammary gland dose calculating unit 105 is the same as that in the first embodiment (FIG. 8A). The contents of basic processing in the mammary gland dose calculating unit 105 according to the second embodiment are the same as those in the first embodiment (FIG. 8B). The following will mainly describe a mammary gland absorbed energy calculating method and an average mammary gland dose calculating method which are different from those in the first embodiment.

A mammary gland absorbed energy calculating method performed by a mammary gland absorbed energy calculating unit 802 according to the second embodiment will be described with reference to the flowchart of FIG. 9 described above. First of all, in step S901, the mammary gland absorbed energy calculating unit 802 performs a radiation imaging simulation with an initial geometric arrangement under simulation conditions. This calculation can use a technique using a Monte Carlo simulation as in the first embodiment. At this time, the mammary gland absorbed energy calculating unit 802 counts the number of photons absorbed in the breast model for each voxel and the energy of each photon. A table 1401 in FIG. 14 shows an example of this counting result. In the table 1401, the rows represent the energies of photons, and the column represents the coordinates of voxels. The second embodiment exemplifies a case in which energies are counted in increments of 10 keV. However, it is preferable to count energies in units corresponding to necessary accuracy. For example, counting may be performed in increments of 1 keV. In addition, the table 1401 shows the result obtained from eight voxels. In practice, however, a result corresponding to the voxel count set in a breast model is output.

In step S902, the mammary gland absorbed energy calculating unit 802 checks whether simulations corresponding to a projection count have been performed. If simulations corresponding to a projection count have been performed, the process advances to step S904; otherwise, the process advances to step S903. In step S903, the mammary gland absorbed energy calculating unit 802 updates the geometric arrangement in accordance with the simulation conditions 801. This processing is the same as that in the first embodiment.

In step S904, the mammary gland absorbed energy calculating unit 802 calculates the sum of energies absorbed by the mammary gland based on equation (5) by using the counted photon counts and their energies. Note that when the energy of each photon has a range as in the table 1401 in FIG. 14 (10 keV in the case shown in FIG. 14), for example, the median value of the range (when the range is 0 keV to 9 keV, the median value is 4.5 keV) is used as the energy of the photon. F_{gsim} as the sum of the energies of radiation absorbed by the mammary gland is defined by

F_{gsim}=∫∫E×N(E,I)×R(E,I)dEdI

where E is the energy of a photon, I is a voxel position, and N(E, I) is the number of photons absorbed by each voxel for each energy. In addition, R(E, I) is a coefficient corresponding to the mammary gland density of a voxel, and is calculated based on equation (6):

$$R ( E , I ) = \frac{{μ}_{breast} ( E ) + {r}_{breast} ( I )}{{μ}_{breast} ( E ) \times {r}_{breast} ( I ) + {μ}_{adipose} ( E ) \times ( 1 - {r}_{breast} ( I ) )}$$

where E is the energy of a photon, I is a voxel position, μ_{breast}(E) is the linear attenuation coefficient of the mammary gland for each energy, μ_{adipose}(E) is the linear attenuation coefficient of the adipose for each energy, and r_{breast}(I) is the mammary gland density of a target voxel.

In step S905, the mammary gland absorbed energy calculating unit 802 calculates the mammary gland dose D_{gsim} from the sum of the energies of radiation absorbed by the mammary gland according to equation (7):

$${D}_{gsim} = α \times \frac{{F}_{gsim}}{{M}_{gsim}}$$

where F_{gsim} is the sum of the energies of radiation absorbed by the mammary gland, M_{gsim} is the mass of the mammary gland, and α is a coefficient for the conversion of a unit into [mGy]. For example, when the unit of F_{gsim} is [keV] and the unit of M_{gsim} is [kg], α is 1.60218×10⁻¹³. The mass M_{gsim} of the mammary gland may be multiplied by a mammary gland density (the ratio between the mammary gland and the adipose) for each voxel to obtain the volume of the overall mammary gland, and the volume may be multiplied by a mammary gland density [g/cm³].

As described above, performing the processing from step S901 to step S907 makes it possible to calculate a three-dimensional model of the mammary gland upon reducing tomographic images and obtain a mammary gland dose. Reducing the breast model from tomographic images in this manner can speed up a mammary gland dose calculation.

### Modification 2-1

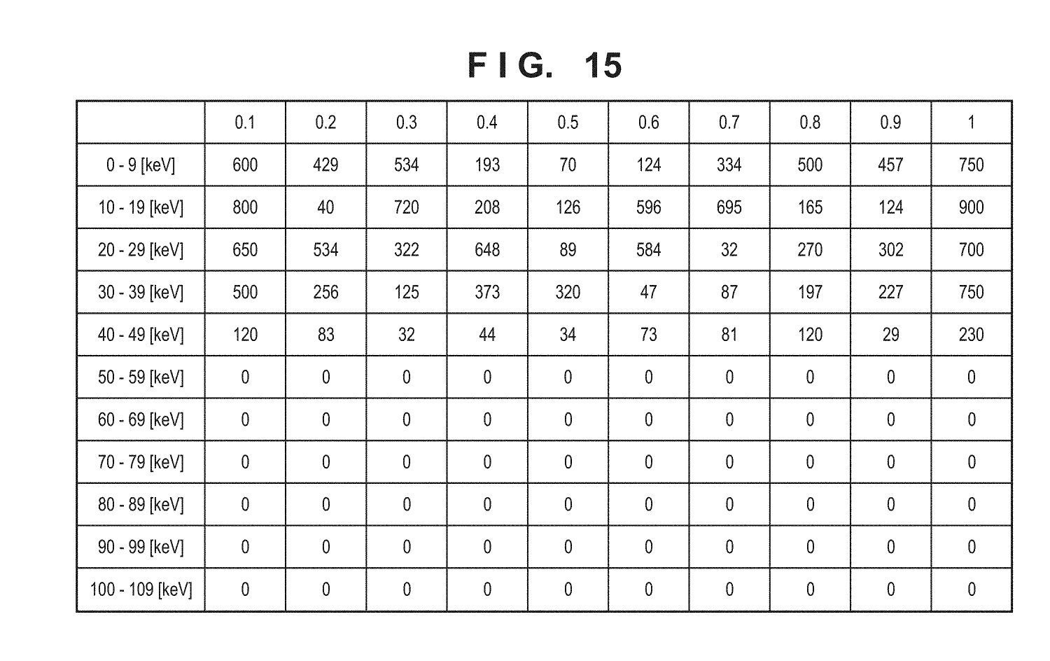

In the second embodiment, no limitation is imposed on the step size of mammary gland densities calculated by the substance information conversion unit 203. Modification 2-1 will exemplify a case in which the step size of mammary gland densities is set to 10% (0.1). The substance information conversion unit 203 decides a mammary gland density corresponding to the energy of radiation and a pixel value. Because mammary gland densities are set in increments of 10%, a mammary gland density is decided depending on to which mammary gland density value the pixel value of a target voxel is closest. Setting mammary gland densities in advance by using a preset step size allows the mammary gland absorbed energy calculating unit 802 to count the energies absorbed by the mammary gland for each mammary gland density and totalize the energy count for each mammary gland density. FIG. 15 shows an example of a counting result. FIG. 15 shows an example of the counting result obtained when the energies of photons are set in increments of 10 keV, and the mammary gland densities are set in increments of 10%.

Totaling energies for each mammary gland density in the above manner eliminates the necessity to hold a result for each voxel. In addition, the sum F_{gsim} of energies absorbed by the mammary gland can be calculated according to equation (8):

F_{gsim}=∫∫E×N(E,r)×R(E,r)dEdr

where E is the energy of a photon, r is a mammary gland density, N(E, r) is the number of photons absorbed for each energy and each mammary gland density, and R(E, r) is a coefficient corresponding to a mammary gland density. The coefficient R(E, r) is calculated according to equation (9):

$$R ( E , r ) = \frac{{μ}_{breast} ( E ) + r}{{μ}_{breast} ( E ) \times r + {μ}_{adipose} ( E ) \times ( 1 - r )}$$

where E is the energy of a photon, r is a mammary gland density, μ_{breast}(E) is the linear attenuation coefficient of the mammary gland for each energy, and μ_{adipose}(E) is the linear attenuation coefficient of the adipose for each energy.

Totaling energies for each mammary gland density in this manner makes it possible to calculate absorbed energies for each mammary gland density and speed up calculation.

### Modification 2-2

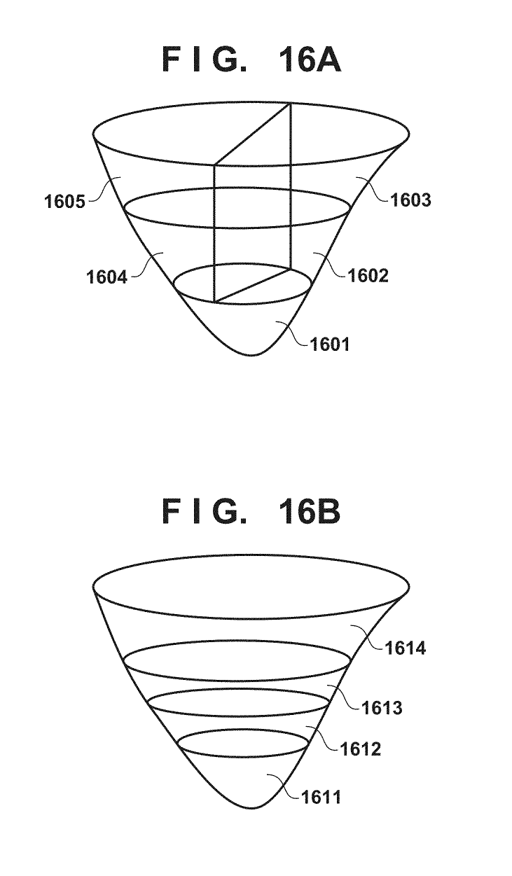

The second embodiment has exemplified the method of reducing voxels when generating a breast model. It is also possible to divide a breast model into areas. In modification 2-2, the breast model generation unit 102 divides a breast model into a plurality of partial areas and decides a mammary gland density (for example, a ratio between the mammary gland and the adipose) based on the average value of voxel values in each of the plurality of partial areas. The mammary gland dose calculating unit 105 then calculates the absorbed dose of the mammary gland for each partial area based on a radiation absorbed dose for each partial area and a mammary gland density for each partial area. FIG. 16A shows a case in which a breast model is divided into seven partial areas. FIG. 16B shows a case in which a breast model is divided into four partial areas in the chest wall direction from the nipple. In the case shown in FIG. 16A, the length in the chest-wall nipple direction is equally divided into three areas, and the two areas located on the chest wall side each are divided into two areas in the craniocaudal direction to generate partial areas 1601 to 1607. In the case shown in FIG. 16B, the length in the chest-wall nipple direction is equally divided into four areas to generate four partial areas 1611 to 1614.

In modification 2-2, the mammary gland dose calculating unit 105 calculates a mammary gland density based on an average pixel value for each of these areas, and calculates the energy absorbed by the mammary gland. Dividing a model in this manner can speed up calculation. In addition, as a mammary gland dose in the second embodiment, an average mammary gland dose of the overall breast is output. However, a mammary gland dose of each of the plurality of partial areas can be output. Outputting a mammary gland dose for each area in this manner makes it possible to check, for example, which area exhibits the highest mammary gland dose. Note that calculating a mammary gland density for each partial area can perform mammary gland component classification in accordance with the mammary gland density of each partial area (for example, a ratio between the mammary gland and the adipose). For example, mammary gland components can be classified into “almost entirely fat”, “scattered fibroglandular densities”, “heterogeneously dense”, “extremely dense”, and the like and output the classification result.

### Modification 2-3

In the second embodiment, mammary gland densities are calculated with respect to reduced tomographic images. However, it is possible to generate a breast model by calculating mammary gland densities from the pixel values of tomographic images that are not reduced.

### Another Modification 1

In the first and second embodiments, mammary gland doses are calculated. However, using a similar simulation, the absorbed dose of other tissues, for example, incident skin doses, can be output. In this case, the breast model generation unit generates a breast model including a three-dimensional model of a skin area as a three-dimensional model of a specific tissue area. A radiation imaging simulation is then performed based on the three-dimensional model and imaging conditions. With this simulation, a skin absorbed dose coefficient indicating how much radiation is absorbed by the skin with respect to an air kerma is calculated by counting energies absorbed by the skin. A skin dose can be calculated by multiplying the actually measured skin absorbed dose coefficient obtained in this manner with the air kerma.

### Another Modification 2

The first and second embodiments each have exemplified the breast imaging apparatus. However, this is not exhaustive. This apparatus may be provided as a dose calculation apparatus that calculates the absorbed dose of a specific tissue in a predetermined region of an object (for example, an apparatus that is incorporated in a breast tomography apparatus and calculates a mammary gland dose or skin dose). In this case, the dose calculation apparatus obtains a tomographic image of an object and imaging conditions at the time of obtaining the tomographic image from a storage unit 1710. The dose calculation apparatus generates a three-dimensional model with a three-dimensional area of a specific tissue of an object and a three-dimensional area other than the specific tissue being discriminated based on tomographic images. The dose calculation apparatus calculates the dose of radiation absorbed by the specific tissue based on the obtained imaging conditions and the generated three-dimensional model.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-174246, filed Sep. 11, 2017, which is hereby incorporated by reference herein in its entirety.

What is claimed is:

1. A breast imaging apparatus that obtains a tomographic image of a breast of an object, the apparatus comprising:
an obtaining unit configured to obtain an imaging condition when the tomographic image is obtained;
a generating unit configured to generate a breast model including a specific tissue with respect to the breast of the object; and
a calculating unit configured to calculate a dose of radiation absorbed by the specific tissue by a radiation imaging simulation using the imaging condition and the breast model.

2. The apparatus according to claim 1, wherein the specific tissue is a mammary gland, and
the calculating unit calculates a mammary gland dose.

3. The apparatus according to claim 2, wherein the calculating unit calculates a mammary gland dose coefficient for conversion of an air kerma into an average mammary gland dose.

4. The apparatus according to claim 2, wherein the generating unit generates a three-dimensional model of the specific tissue by using voxels, of voxels constituting a three-dimensional tomographic image based on the tomographic image, which are classified as mammary gland voxels based on voxel values.

5. The apparatus according to claim 4, wherein the generating unit classifies voxels in the breast into mammary gland voxels and adipose voxels based on voxel values.

6. The apparatus according to claim 4, wherein the generating unit classifies a voxel as one of a mammary gland voxel, an adipose voxel, a skin voxel, an implant voxel, and a calcification voxel.

7. The apparatus according to claim 2, wherein the generating unit generates the breast model by using voxels classified as mixture voxels of the mammary gland and the adipose based on voxel values, and
the calculating unit calculates an absorbed dose of the mammary gland for each voxel based on a radiation absorbed dose in voxels constituting the breast model and a mammary gland density decided based on voxel values.

8. The apparatus according to claim 2, wherein the generating unit divides the breast model into a plurality of partial areas and decides a mammary gland density based on an average value of voxel values for each of the plurality of partial areas, and
the calculating unit calculates an absorbed dose of the mammary gland for the each partial area based on a radiation absorbed dose for the each partial area and the mammary gland density for the each partial area.

9. The apparatus according to claim 7, wherein the generating unit sets the mammary gland density by using a predetermined step size.

10. The apparatus according to claim 2, wherein the generating unit generates a breast model including a mixture of the mammary gland and the adipose based on values of voxels after reduction of the tomographic image.

11. The apparatus according to claim 2, further comprising an output unit configured to overlay and display a mammary gland dose distribution calculated by the calculating unit on the tomographic image.

12. The apparatus according to claim 8, further comprising an output unit configured to output a mammary gland dose for each of the plurality of partial areas.

13. The apparatus according to claim 7, wherein the calculating unit counts energies absorbed by the mammary gland for each mammary gland density.

14. The apparatus according to claim 3, wherein the calculating unit calculates an average mammary gland dose from the mammary gland dose coefficient and a dose value obtained by an area dosimeter.

15. The apparatus according to claim 2, wherein the calculating unit calculates the mammary gland dose upon excluding a designated portion from a breast model.

16. The apparatus according to claim 1, wherein the specific tissue is a skin.

17. The apparatus according to claim 16, the calculating unit calculates a skin absorbed dose coefficient indicating a ratio of a dose of radiation absorbed by the skin to an air kerma by performing a radiation imaging simulation based on the breast model and the imaging condition.

18. The apparatus according to claim 8, further comprising a classifying unit configured to perform mammary gland component classification in accordance with a mammary gland density for the each partial area.

19. The apparatus according to claim 1, wherein the calculating unit performs a simulation with a projection count smaller than a projection count at time of breast tomography.

20. A dose calculating apparatus comprising:
an obtaining unit configured to obtain a tomographic image of an object and an imaging condition when the tomographic image is obtained;
a generating unit configured to generate a three-dimensional model with a three-dimensional area of a specific tissue of the object and a three-dimensional area other than the specific tissue being discriminated; and
a calculating unit configured to calculate a dose of radiation absorbed by the specific tissue based on the imaging condition and the three-dimensional model.

21. A control method for a breast imaging apparatus that obtains a tomographic image of a breast of an object, the method comprising:
obtaining an imaging condition when the tomographic image is obtained;
generating a breast model including a three-dimensional model of a specific tissue with respect to the breast of the object; and
calculating a dose of radiation absorbed by the specific tissue by a radiation imaging simulation using the imaging condition and the breast model.

22. A dose calculating method comprising:
obtaining a tomographic image of an object and an imaging condition when the tomographic image is obtained;
generating a three-dimensional model with a three-dimensional area of a specific tissue of the object and a three-dimensional area other than the specific tissue being discriminated; and
calculating a dose of radiation absorbed by the specific tissue based on the imaging condition and the three-dimensional model.

23. A non-transitory computer-readable medium storing a program for causing a computer to execute a control method for a breast imaging apparatus configured to obtain a tomographic image of a breast of an object, the control method comprising:
obtaining an imaging condition when the tomographic image is obtained;
generating a breast model including a three-dimensional model of a specific tissue with respect to the breast of the object; and
calculating a dose of radiation absorbed by the specific tissue by a radiation imaging simulation using the imaging condition and the breast model.

24. A non-transitory computer-readable medium storing a program for causing a computer to execute a dose calculating method, the dose calculating method comprising:
obtaining a tomographic image of an object and an imaging condition when the tomographic image is obtained;
generating a three-dimensional model with a three-dimensional area of a specific tissue of the object and a three-dimensional area other than the specific tissue being discriminated; and
calculating a dose of radiation absorbed by the specific tissue based on the imaging condition and the three-dimensional model.
